(19) United States
(12) Patent Application Publication
(10) Pub. No.: US 2021/0308071 A1
(43) Pub. Date: Oct. 7, 2021

(54) Treatment method to abort and or reduce the severity of upper respiratory viral illness as well as to prevent upper respiratory viral illness in humans

(71) Applicant: Anantha Lakshmi Thallapureddy, Austin, TX (US)

(72) Inventor: Anantha Lakshmi Thallapureddy, Austin, TX (US)

(21) Appl. No.: 17/221,838

(22) Filed: Apr. 4, 2021

Related U.S. Application Data
(60) Provisional application No. 63/005,306, filed on Apr. 4, 2020.

(51) Int. Cl. A61K 31/045; A61K 33/14; A61K 9/00; A61K 36/9066; A61K 31/12; A61K 36/67; A61K 33/30; A61K 31/375; A61K 36/28; A61K 36/185; A61K 36/8962; A61K 36/61; A61K 36/53; A61K 36/9068

(52) U.S. Cl. CPC A61K 31/045; A61K 9/0056; A61K 9/006; A61K 36/9066; A61K 31/12; A61K 36/67; A61K 33/30; A61K 31/375; A61K 36/28; A61K 36/185; A61K 36/8962; A61K 36/61; A61K 36/53; A61K 36/9068; A61K 33/14

(57) ABSTRACT

Upper respiratory viral illness including influenza is a common worldwide problem. The consequences of these infections in any given population have a huge spectrum including losing several workdays all the way to severe illness with prolonged hospitalizations and death. While there are a wide variety of symptomatic treatments available, they are neither shown to alter the disease course nor prevent the occurrence of secondary complications. There are no options available to get rid of these infections before the infection becomes a full blown illness. After various preventive methods like vaccines, hand hygiene, wearing masks and avoiding exposure, the next best step is to make use of treatment methods that do not necessitate a doctor visit or expensive diagnostic tests, to get rid of the illness quickly. The inventor here describes an invention of a treatment method to abort and/or reducing the severity either in its duration or symptoms or both of upper respiratory viral illness using a unique gargling solution comprising of Menthol, Borneol and Sodium Chloride dissolved in warm water, wherein the three compounds work in synergy for the above mentioned purpose.

As I continued researching other additional ways to further improve outcomes in humans with upper respiratory viral illness, I also formulated an additional treatment method to create a multi target therapy. This additional oral treatment to be used in conjunction with the above gargling method to ensure the best outcome for everyone with the said illness but particularly for individuals whose immune system may be already stressed. This oral formulation is a carefully formulated mixture of Curcumina Longa root powder along with Curcumin extract, Piper Nigrum, Ascorbic acid, Zinc, Echinacea, Sambucus Nigra, Allium Sativum, Syzygium Aromaticum, Coleus Amboinicus and Zingiber Officinale to be taken orally.

### CROSS-REFERENCE TO RELATED APPLICATION

This application claims the benefit of priority to U.S. provisional application No. 63/005,306 filed on Apr. 4, 2020, entitled “An alternative treatment to abort and or reduce the severity of upper respiratory viral illness in humans.” That provisional application is incorporated herein by reference for all purposes.

### PRIOR ART DESCRIPTION

Many ointments and creams containing Menthol and Camphor like Vicks Vaporub are available as a local rub to be applied on the skin of chest, front of the neck and upper back to relieve symptoms of cold and congestion but they do not exert any direct effect on the upper airway mucosa and does not work to abort and/or reduce the severity of upper respiratory viral illness or prevent the same. Moreover, due to prolonged contact with skin, there is a higher chance of absorbing these ingredients that can lead to an excessive cumulative dose. These preparations do not make use of the synergistic antimicrobial effect of Sodium chloride. The same is true for inhalation preparations containing Menthol.

There are also cough drops and lozenges with Menthol and/or Camphor. The issues with these formulations are that it involves ingestion of these ingredients which have shown to cause various adverse reactions, they do not make use of Sodium chloride's antimicrobial effects and they do not abort the said illness or reduce its severity significantly. In our treatment method, the active ingredients are in contact with mucosal surfaces for a very brief period of time making it much safer.

Some of the prior art used Iodine as the active ingredient. But at a concentration that is effective as an antimicrobial particularly as an antiviral gargle, it has shown to cause mucosal membrane disruption and damage, hence that method is not recommended.

The prior art publication, numbered JP2004051511A, describes using an ammonium salt type fungicide and Dipotassium glycyrrhizinate. Ammonium salts like ammonium chloride at concentrations safer as a gargle are mainly known to work as an expectorant by increased mucus production due to its irritating effect on the bronchial mucosa. Dipotassium glycyrrhizinate now is shown to inhibit 11-BETA-HYDROXYSTEROID DEHYDROGENASES and other enzymes involved in the metabolism of CORTICOSTEROIDS causing hypermineralocorticoidism with sodium retention and potassium loss, edema, increased blood pressure, as well as depression of the renin-angiotensin-aldosterone system.

In the U.S. Ser. No. 12/466,537, the active ingredients are menthol and Hydroxyethylrutoside and the later ingredient is a semisynthetic flavonoids, which acts primarily on the microvascular endothelium to reduce hyperpermeability and edema. For a gargling solution to effectively abort and/or reduce the severity of an upper respiratory infection, it has to work as an efficient antiviral agent capable of combating the very early phase of the viral entry in to epithelial cells and its replication before such hyperpermeability, inflammation and edema occurs. More over the said agent is currently used mainly to improve microvascular disruption in wounds and for poor circulation issues.

Few other publications describe various herbs but not any particular herbal extract in concentrated form like menthol, camphor or borneol as a mouthwash needing to apply 1 tablespoon of the contents to the oral cavity 6 times per day with first 2 doses being 2 hours apart. The practicality and lack of sufficient concentration of the said antimicrobial agents in such formulations can limit its ability to abort an upper respiratory viral illness.

### Some of the Advantages of My Invention

None of the existing topical treatment methods use Borneol. Borneol has 2 advantages. One is that it is known to selectively inhibit proinflammatory Transient Receptor Potential (TRP) ion channel TRPA1 there by combating the irritative effects of menthol, but non affecting menthol's other very important beneficial effects to fight the said infection. Secondly, Borneol's metabolism in humans is shown to be much simpler compared to Camphor making it much safer. All the ingredients in this treatment method have a well established safety profile. None of the other gargle treatment methods are said to give an additional benefit of an immediate symptomatic relief. None of the other gargle treatment methods combines additional oral formulation that has the highest safe doses of Curcumina Longa Rhizome extract, Vitamin C and Zinc along with various other ingredients described in the oral treatment method below.

### BACKGROUND OF THE INVENTION

The morbidity and mortality from upper respiratory viral illness are enormous in the USA. When we look at influenza alone, CDC estimated that from Oct. 1, 2019, to Mar. 7, 2020, there were 36 to 51 million cases, 370,000 to 670,000 hospitalizations and 22,000 to 55,000 deaths from influenza. In 2017, the economic burden of influenza in the US was estimated to be 10.4 billion dollars. In addition to the above said consequences, there is yet another major health burden to look at. A well known complication of upper respiratory viral illness is the development of secondary bacterial infections like bronchitis/sinusitis/otitis media/pneumonia, needing antibiotics. According to a CDC report in 2017, 47 million antibiotics were given unnecessarily and 50% of which were given for upper respiratory infections. As we know very well, the burden of unnecessary antibiotic usage is enormous including Clostridium difficile infections, antibiotic resistance, antibiotic side effects, drug interactions and even more important is, destruction of the healthy gut bacteria called probiotics. Current medical research shows that altered gut flora and other normal human flora can lead to several chronic illnesses, both directly and indirectly. Upper respiratory viral illness also leads to COPD exacerbation often requiring steroid use in addition to antibiotics, in patients who already have HTN, Diabetes etc, causing significant short term and long term side effects.

As a board certified MD practising medicine in the United States since 2003, I could clearly see the burden of upper respiratory viral illness and now COVID-19 in the US and the lack of effective treatments. Undoubtedly vaccines are the best way to lessen the morbidity and mortality but it has its own limitations. For example, the influenza vaccine's efficacy varies season to season depending on how aggressively the virus mutates and it may be hard to vaccinate one hundred percent of the population. Influenza medications are effective only within 48 hours of illness onset, they generally do not abort the illness, and there is evidence of developing antiviral resistance. Also, most antiviral drugs have many side effects. Moreover except influenza, all the other upper respiratory viruses do not have any proven antiviral medications yet. There are many symptomatic treatments available but none of them can get rid of the infection or even reduce the severity and duration of the infection. Many of the symptomatic treatments have their own side effects. For example, decongestants can raise blood pressure and even non sedating antihistamines can cause drowsiness and dry mouth. Also, drug interactions are always a concern in the elderly taking multiple medications already. In terms of direct prevention, apart from hand hygiene methods and avoiding exposure, no other preventive methods are available at this point.

In order to protect myself and my family from upper respiratory viral illness, I have looked into every possible method for several years. I aimed to reduce the severity of an upper respiratory illness and if possible to get rid of it before it becomes a full blown illness. I am always particularly interested in prevention and lean towards treatments with the least side effects. Saltwater gargling is a known method but it only helps to ease just the throat symptoms for a brief period. Studies showed overall only a minor benefit of such method (6) In addition, few other treatments use Menthol as a local rub, inhalation or lozenge all of which gives brief symptomatic relief but does not get rid of the infection or reduce the severity of the infection.

After I continued to experiment with different ingredients over a period of time, I ended up adding Borneol, Menthol and Sodium chloride to water heated to between 95 to 130 degrees Fahrenheit to make a gargling solution and I experimented a few times using different amounts of each compound and finally came up with a mixture that is effective but not too strong in terms of flavor. To my delight, this became a very effective treatment method for my family, extended family and our friends, to lessen the suffering from the upper respiratory viral infections. As I continued to use it I realized that if the method is used very early in the disease course, it will abort the illness altogether leading to no further progression of the illness. In addition to above said results it also gives a quick symptomatic relief as well. Based on basic science publications about the cytoprotective and anti-inflammatory effects of Menthol and Borneol, I understood the underlying powerful synergistic effects of all the ingredients combined, producing a robust antiviral effect in the oro-pharyngeal epithelial cells. By continuing to experiment, I learned that this method can prevent upper respiratory viral illness if used soon after a deemed exposure to such illness.

Due to the COVID 19 pandemic, I continued researching further to improve outcomes in humans with upper respiratory viral infections and formulated additional treatment to create a multi target therapy. This additional oral treatment should be used preferably in conjunction with the above said gargling solution but also can be used by itself, to ensure the best outcome for all especially to those whose immune system may be already stressed and for those who may not be able to do an effective gargle. This oral formulation is a carefully formulated mixture of Curcumina longa root powder along with Curcumin extract, Piper nigrum, Ascorbic acid, Zinc, Echinacea, Sambucus nigra, Allium sativum, Syzygium aromaticum, Coleus amboinicus and Zingiber Officinale to be taken orally.

### SUMMARY OF MY INVENTION

In summary, this invention is about a treatment product and a method of aborting and/or reducing the severity as well as preventing upper respiratory viral illness in humans. The product comprises of an innovative, safe and effective topical gargling solution comprising of Menthol and Borneol each in the range of 0.01 gm to 0.15 gm and Sodium chloride in the range of 1 to 4 gm mixed in 4 to 8 ounces of water that is heated to a temperature between 95 to 130 degrees Fahrenheit. This method of treatment is to administer the said product to gargle the oro-pharyngeal region of a person with such infection by pushing the medicine into the back of the throat and thoroughly swishing with the solution for 5 to 30 seconds and spit it out and repeats it for 2 to 3 more times. The person will start this method at the onset of the very first symptom of the said illness and will repeat it 1-2 more times in the first 24 hours, preferably 6-8 hours apart and another 1 to 2 times in the next 24 hours. The treatment method also gives a quick symptomatic relief from the said illness. The above said gargling mixture should be used within 1-2 minutes after mixing with water.

For preventing an upper respiratory viral infection, the subject will be using the gargling treatment and method said above soon after a deemed exposure to said illness and preferably to be repeated one more time in 4 hours.

Aborting the infection means getting rid of the infection in its early stages so that it will not progress further.

Reducing the severity of the infection means either reducing the severity of the symptoms or duration of the illness or both.

Above said treatment method will be followed by an oral formulation containing a carefully formulated mixture of Curcumina longa root powder along with Curcumin extract, Piper nigrum, Ascorbic acid, Zinc, Echinacea, Sambucus nigra, Allium sativum, Syzygium aromaticum, Coleus amboinicus and Zingiber Officinale to be taken orally twice a day for 2-3 days and then once a day for another 3-7 days

To my knowledge, there is no such effective and safe topical treatment method available to date that can actually abort and/or reduce the severity of the above said illness along with giving immediate symptomatic relief as well as be useful as a prophylactic method to prevent the onset of said illness.

Combining above said innovative and highly effective gargling mixture with an oral formulation comprising the highest safe dose of various highly potent anti-inflammatory, antioxidant, antiviral, anti-apoptotic, anti nausea, antipyretic, anti fatigue as well as antitussive compounds as a multi-defense strategy also makes this innovation novel.

### DETAILED DESCRIPTION OF THE INVENTION

This invention makes use of several different ingredients that work in synergy to exert their anti-inflammatory, antimicrobial and antioxidant effects. The following scientific studies give a great insight into these mechanisms.

As part of the multi defensive therapy, an oral formulation comprising the highest safe dose of various highly potent anti inflammatory, antioxidant, antiviral, anti-apoptotic, anti nausea, antipyretic, anti fatigue and antitussive compounds is designed as well and it is described below.

The following is a brief introduction to the active ingredients:

### Menthol (C10H20O)

Menthol is an organic compound found in various mint plants and is well known for its medicinal effects. Studies have shown that Menthol activates various Transient Potential Receptor (TPR) ion channels like TRPV1 and TRPM8 and even to some extent the TRPA1 channels. Its effects are stronger on TRPM8. These channels are present in the somatosensory nerve endings as well as the epithelial cells of the upper and lower respiratory system. Studies showed that lipophilic Menthol effectively gets access to the nerve endings below the multilayered squamous epithelium and exert its cooling effect without lowering the temperature via binding to TRPM8 (1). Another study showed that after activation of TRPM8 channels of the rat peritoneal macrophages, enhanced phagocytic activity and anti-inflammatory cytokine activity was demonstrated (5). Ariane Leite Rozza et al in their study showed that oral treatment with Menthol displayed a gastroprotective activity through anti-apoptotic, antioxidant and anti-inflammatory mechanisms (4). Zhang et al showed that Menthol opens the tight junctions and weakens the barrier capabilities of epithelial cells, thereby promoting the permeability of puerarin in their study (3). This is a likely mechanism where Menthol can make the Sodium chloride and Borneol reach deeper infected cell layers. Menthol is also shown to cause increased local mucin production in the upper respiratory mucosa (2) which will further augment the antiviral defense(X) and also shown to increase thermogenesis in white adipose tissue (7).

### Borneol (C10H18O)

Borneol is a highly lipid-soluble bicyclic monoterpene with fragrant odor and pungent tastes. Borneol occurs naturally in over 260 plants and is found in citrus peel and thyme oils, spices such as nutmeg, ginger and thyme. It is jointly approved by FAO/WHO experts as a food additive. Borneol is shown to inhibit the pro-inflammatory ion channel TRPA1 (8). Various studies reported that Borneol has neuroprotective effects through a variety of mechanisms like antinociception (10, 14), anti-inflammatory (15, 16), antioxidation (16), and anti-epilepsy properties (17). One important benefit of Borneol compared over Camphor is that its metabolism and elimination are simpler than Borneol and the description in the journal produced after the Sixty-third meeting of the Joint FAO/WHO Expert Committee on Food Additives (JECFA) says, the bicyclic secondary alcohols like Borneol are rapidly conjugated with glucuronic acid in humans, dogs, and rabbits and excreted via the urine. In humans (FIG. 5), 81% and 94% of the orally administered dose of Borneol at 1000 and 2000 mg, respectively, were excreted as the glucuronic acid conjugate within 24 hours. At 10 hours after ingestion of 2000 mg of Borneol, 81% of the administered dose was detected as the glucuronic acid conjugate in human urine

### Sodium Chloride

The antiviral property of Sodium chloride is due to the chloride part that is used by the cells to form hypochlorous acid (HOCL) which has well known antimicrobial activity.

In summary, Menthol along with Sodium chloride and Borneol works in synergy, to abort and/or reduce the severity of an upper respiratory viral illness by various proposed mechanisms. As an example, enabling Sodium chloride to reach deeper layers of epithelial cells to exert its antimicrobial activity, inducing mucin production, transient vasodilatation, inducing anti-inflammatory cytokines and reducing pro-inflammatory cytokines. Borneol in addition to its anti-inflammatory and anti-apoptotic effects lessens the mucosal irritating effect of Menthol by antagonizing the Menthol induced TRPA1 activation and possibly reducing Menthol induced CNS stimulation, via its known neuroprotective activity. Altogether, this treatment method can lead to a quick and robust local defense mechanism.

The composition of the mixture of the 3 active ingredients results in slightly oily granular powder and the final mixture in warm water is granular and colored pale yellow

I have also been successful in employing this treatment method as a prophylaxis for subjects soon after they deemed to be exposed to the said illness.

The said additional oral treatment method is used along with the topical gargle therapy as a part of a multi-target/multi-defense mechanism to abort and or reduce the severity of upper respiratory viral illness in humans. The advantages of this combined multi-target/multi-defense therapy are as follows:
1. It has the highest safe dose of Curcumina longa.
2. It has the highest safe dose of Zinc.
3. It has an ingredient with the highest antioxidant nature.
4. It has an antitussive and expectorant.
5. It has a gastro protective agent to help with nausea and stomach upset that is commonly experienced in subjects with active upper respiratory viral illness.
6. By using this oral formulation along with the unique gargling solution that is described above, the chances of aborting and/or reducing the severity of upper respiratory viral infection significantly enhanced, particularly for people with already stressed immune systems due to multiple comorbidities.

It is time to maximize the applicability of all possible prevention and treatment methods, particularly with agents with minimal to no side effects, for upper respiratory viral infections in humans. Having the two methods described above be available together as a dual therapy will improve effectiveness and compliance and reduce confusion for the consumer.

Following is a brief description of the ingredients in the oral formulation

### 1. Curcumina Longa Root Powder Combined with Curcumin Extract

It is a well known phytochemical with a multi mechanistic mode of action and commonly used culinary herb. It is an FDA approved herbal medicine. Curcumin is more active for arthritic pain and inflammation relief but for antiviral purposes, the whole Curcumina longa root powder is equally important as its extract Curcumin. Also, it works as an antipyretic and anti-fatigue formula as well.

Fatemeh et al in their peer reviewed publication summarized Some of Curcumin's potent antiviral mechanisms including COVID-19 infection (y). In addition, its safety is very well established even at as high as 8-12 gm/day

a) Curcumin modulates intercellular signalling cascades which are essential for efficient virus replication

b) Curcumin also affects cellular post-transcriptional and post-translational modifications, thereby limiting the viral multiplication by interfering with crucial steps in their replication cycle

c) Curcumin treatment can modify the structure of the surface protein in viruses, thereby blocking the entry of virus and virus budding.

d) Curcumin may have beneficial effects against COVID-19 infection via its ability

to modulate the various molecular targets that contribute to the attachment and internalization of SARS-CoV-2 in many organs, including the liver, cardiovascular

system, and kidney.

e) Curcumin could also modulate cellular signalling pathways such as inflammation, apoptosis, and RNA replication.

f) Curcumin may also suppress pulmonary edema and fibrosis-associated pathways in COVID-19 infection

The formulation in the instant invention may comprise Curcuma Longa root powder from 1000 mg to 8000 mg, preferably from 2000 mg to 4000 mg

### 2. Piper Nigrum

Piper nigrum is proved to improve the absorption of Curcumin. It is a thermogenic herb that also helps reduce the progression of cough and is a common culinary herb. The formulation in the instant invention may comprise piper nigrum from 5 mg to 20 mg

### 3. Ascorbic Acid

Ascorbic acid is a well known powerful antioxidant and immunity booster.

The formulation in the instant invention may comprise ascorbic acid from from 250 mg to 750 mg, preferably from 250 mg to 500 mg

### 4. Zinc

Zinc is an essential trace element that is crucial for growth, development, and the maintenance of immune function and is shown to exert a direct antiviral effect, as well as stimulation of antiviral immunity (a)

Multiple clinical studies showed the safety of high dose Zinc in humans (b) The formulation in the instant invention may comprise zinc from 15 to 50 mg, preferably 30 mg to 50 mg.

### 5. Echinacea Purpurea

Several laboratory and animal studies suggest that Echinacea contains active substances that boost immune function, relieve pain, reduce inflammation, and have antiviral and antioxidant effects.

The formulation in the instant invention may comprise Echinacea Purpura extract from 250 mg to 800 mg, preferably from 300 to 500 mg.

### 6. Sambucus Nigra

Its high content of phenolic acid, flavonoids and anthocyanins exert antioxidant effects and later also is a known anti-inflammatory agent. Small scale clinical studies in people with upper respiratory infections showed a faster recovery of the infection in those who used it compared to those who did not.

The formulation in the instant invention may comprise Sambucus Nigra from 500 mg to 1000 mg.

### 7. Allium Sativum

The allicin in this herb has known antimicrobial properties and is used as a common culinary herb.

The formulation in the instant invention may comprise Allium Sativum from 100 mg to 800 mg, preferably from 250 mg to 500 mg.

### 8. Syzygium Aromaticum

This herb is top in the list when it comes to antioxidant capacity (1) with a very high ORAC (Oxygen Radical Absorbing Capacity) of about 300,000 and has many known health benefits and It is used as a culinary herb.

The formulation in the instant invention may comprise Syzygium Aromaticum from 100 to 500 mg, preferably from 150 to 300 mg.

### 9. Coleus Amboinicus

Some of the important phenolic acids present in this herb are caffeic acid, gallic acid, rosmarinic acid and Some of the important flavonoids present are chrysoeriol, cirsimaritin, eriodictyol, luteolin and rutin (Z)

In addition to its antiviral/anti-inflammatory properties, it is a known natural expectorant and helps to relieve coughs and headaches and is also used as a culinary herb.

### 10. Zingiber Officinale

One of the great benefits of Zinger Officinale rhizome is that apart from its anti-inflammatory and antiviral properties, it helps to ameliorate nausea and stomach upset. It has been used as a common culinary herb for centuries.

The formulation in the instant Invention may comprise of zinger officinale rhizome from 250 mg to 750 mg, preferably from 400 to 500 mg.

I claim:

1. A formulation of a gargling mixture comprising of Menthol, Borneol and Sodium Chloride with or without a flavouring agent that is used to abort and/or reduce the severity of an upper respiratory viral illness.

2. The formulation of claim 1 further comprising a liquid to facilitate gargling.

3. The formulation of claim 2 wherein the liquid is water.

4. The formulation of claim 3 wherein Menthol by weight is from 0.025 gm to 0.15 gm.

5. The formulation of claim 4 wherein Borneol by weight is from 0.025 to 0.15 gm.

6. The formulation of claim 5 wherein Sodium chloride by weight is from 1 gm to 3 gm.

7. The formulation of claim 6 wherein mixed into two to eight ounces of water.

8. The formulation in claim 7 wherein water temperature is in a range of 95 to 130 degrees Fahrenheit.

9. A method to abort and/or reduce the severity of upper respiratory viral infections in a subject in need thereof comprising administering the formulation in claim 1 wherein the subject gargles with the said formulation 2-3 times on first day of the said illness, and 1-2 times the second day.

10. A method to prevent an upper respiratory viral infection comprising administering the formulation in claim 1 where in the subject in need thereof gargles with the said formulation one to two times after an occurrence of an exposure to such infection.

11. Wherein the method of claim 9 further comprises of administering an oral formulation comprising of:
Curcuma Longa root powder from 1500 mg to 8000 mg.
Curcumin extract from 100 to 500 mg.
Piper Nigrum from 5 mg to 20 mg.
Zinc from 15 mg to 50 mg.
Ascorbic acid from 250 mg to 750 mg.
Echinacea purpurea extract from 250 mg to 800 mg.
Sambucus Nigra extract from 500 mg to 1000 mg.
Allium Sativum from 200 mg to 800 mg.
Syzygium Aromaticum from 100 mg to 500 mg.
Coleus Amboinicus from 200mg to 800mg
Zingiber Officinale from 250 mg to 750 mg.
A nutritionally acceptable carrier.
